(19) United States
(12) Patent Application Publication
(10) Pub. No.: US 2021/0023170 A1
(43) Pub. Date: Jan. 28, 2021

(54) FVIII CHIMERIC ANTIGEN RECEPTOR TREGS FOR TOLERANCE INDUCTION IN HEMOPHILIA A

(71) Applicant: UNIVERSITY OF FLORIDA RESEARCH FOUNDATION, INCORPORATED, GAINESVILLE, FL (US)

(72) Inventors: Roland Wilfried HERZOG, Gainesville, FL (US); Moanaro BISWAS, Gainesville, FL (US); Todd Michael BRUSKO, Gainesville, FL (US)

(21) Appl. No.: 16/969,353

(22) PCT Filed: Feb. 12, 2019
(86) PCT No.: PCT/US2019/017630

§ 371 Date: Aug. 12, 2020

Related U.S. Application Data
(60) Provisional application No. 62/629,139, filed on Feb. 12, 2018.

(51) Int. Cl. A61K 38/17; C12N 15/867; A61P 7/00; C07K 16/28; A61K 48/00

(52) U.S. Cl. CPC A61K 38/177; C12N 15/867; A61K 48/00; C07K 16/283; A61P 7/00

(57) ABSTRACT

Provided are materials and methods for generating chimeric antigen receptors (CARs) specific for human factor VIII (huF.VIII), which huF.VIII CARs are expressed in regulatory T cells and used to treat inhibitor formation in hemophilia A patients. Further provided are novel huF.VIII proteins and nucleic acids encoding the novel huF.VIII CAR as well as methods to treat inhibitor formation using therapeutically effective amounts of Tregs expressing the novel huF.VIII CAR.

### BACKGROUND OF INVENTION

Hemophilia is the X-linked bleeding disorder caused by mutations in coagulation factor IX (FIX, hemophilia B) or its co-factor, factor VIII (FVIII, hemophilia A).

Since the serine protease FIX has very low activity in the absence of FVIII, mutations in either protein can cause the coagulation defect.

Hemophilia A has a higher prevalence, occurring in about 1:5,000 male births, while hemophilia B occurs in about 1:25,000. The loss of function of either F.VIII or F.IX results in a defect in the intrinsic clotting cascade.

In the intrinsic pathway, exposure of circulating F.XII to a damaged surface causes its activation. Active F.XII (F.XIIa) activates F.XI, which then in conjunction with extrinsically activated tissue factor-F.VIIa complex (extrinsic factor Xase) proceeds to cleave the zymogens F.IX and F.X into their active forms, F.IXa and F.Xa. F.IXa is a serine protease whose function depends on the post-translational γ-carboxylation of F.IX by vitamin K (Rogers et al., 2015).

Meanwhile, activation by the extrinsic pathway also results in cleavage of the glycoprotein F.VIII into activated F.VIIIa. F.VIIIa (cofactor) and F.IXa (enzyme) come together to form the intrinsic factor Xase. This complex cleaves F.X into F.Xa at a rate much higher than the extrinsic factor Xase, such that in the end about 90% of F.Xa is produced by the intrinsic complex. The activity of the intrinsic factor Xase is dependent on binding to phospholipid membranes on endothelial cells or platelets as well as free Ca²⁺. Activated F.Xa facilitates the conversion of prothrombin into thrombin, which then catalyzes the formation of the fibrin clot. Thus, a genetic defect in F.VIII or F.IX prevents the assembly of the intrinsic factor Xase, significantly impairing the ability to activate F.X and induce formation of the fibrin clot.

The severity of X-linked hemophilia is dependent on the degree of residual clotting activity. Mild cases (5-40% clotting activity) are typically asymptomatic outside of major trauma or surgery, whereas moderate cases (1-5% clotting activity) are somewhat more vulnerable, and may evidence prolonged bleeding even from minor injuries. However, severe hemophilia (<1% clotting activity) brings additional complications. In addition to the difficulty responding to injury, these patients frequently develop spontaneous bleeds in capillary beds, particularly within joints. Over time, this causes significant chronic deterioration of the joints if not properly managed. Currently, hemophilia is treated by intravenous delivery of replacement clotting factor, either plasma-derived or recombinant. This therapy can be performed on demand, though it has been suggested that prophylactic management (typically 3 injections per week) can reduce joint damage over time.

As patients are not naturally producing clotting factor, the immune system can recognize the exogenous protein as a foreign antigen and form antibodies against the protein that prevent its function; these neutralizing antibodies are also known as “inhibitors”. The frequency of inhibitor formation varies by disease: about 25-30% of hemophilia A, but only about 5% of hemophilia B patients develop inhibitors. The risk for inhibitor formation varies depending on a number of factors, including the severity of the underlying mutation; both preclinical and clinical studies indicate that more residual protein expression reduces inhibitor formation in both hemophilia A and B (Markusic et al., 2013; Chitlur et al., 2009; Gouw et al., 2012; Halimeh et al., 2013; Mariani et al., 2003). The development of inhibitors against coagulation factor VIII (F.VIII) is a critical complication in the treatment of hemophilia A, as hemostasis can no longer be re-established by F.VIII replacement.

Inhibitor formation is mostly dependent on CD4+ T helper cells, leading to B cell activation, class-switching to IgG, and generation of memory B cells and antibody-producing plasma cells. Induction of CD4+ T cell tolerance represents a potential approach to prevent or reverse inhibitor formation.

Inhibitors seriously complicate treatment and increase morbidity and mortality of hemophilia. Currently, the only treatments for inhibitor formation are immune tolerance induction (ITI) protocols. Increased factor doses are used and may be able to restore hemostasis in patients with low-titer inhibitors (<5 Bethesda Units, BU), while F.VIII bypassing factors exhibiting sufficient F.VIII-independent activity are required to treat a bleed in the presence of high-titer inhibitors. However, these treatments are expensive and have to be carefully dosed. Clinical protocols for reversal of the antibody response via immune tolerance induction (ITI) consist of frequent high-dose factor administrations for prolonged periods (months to >1 year), are very expensive (>S1,000,000), and ˜30% of F.VIII inhibitor patients fail to respond.

Therefore, new and improved tools are needed that can work alone or in conjunction with prolonged ITI or bypassing agents to improve hemostasis in patients that have failed ITI.

### BRIEF SUMMARY

The subject invention provides methods and materials to engineer novel chimeric antigen receptors (CARs) that combine the antibody specificity with T regulatory function without the need for MHC restriction and are used for tolerance induction in hemophilia A. Also provided are regulatory T cells expressing the novel CARs of the invention and methods for using the novel CAR expressing Tregs for tolerance induction in hemophilia A.

The CARs of the subject invention comprise a single chain antibody variable region (scFv) derived from an antibody produced in a B cell of a hemophilia patient who has received human factor VIII (huF.VIII) protein therapy and has developed antibodies to huF.VIII protein (huF.VIII inhibitors). The novel CARs further comprise a CD28 signaling domain and a CD3 signaling domain, the latter being engineered for optimized functionality of the huF.VIII CAR in Treg cells.

### BRIEF DESCRIPTION OF DRAWINGS

The patent or application file contains at least one drawing executed in color. Copies of this patent or patent application publication, with color drawing(s), will be provided by the Office upon request and payment of the necessary fee.

FIG. 1 is a diagram depicting the activation of F.VIII-specific B cells upon interaction with CD4+ T helper cells leading to inhibitor formation and the suppression of inhibitor formation at various stages by Tregs.

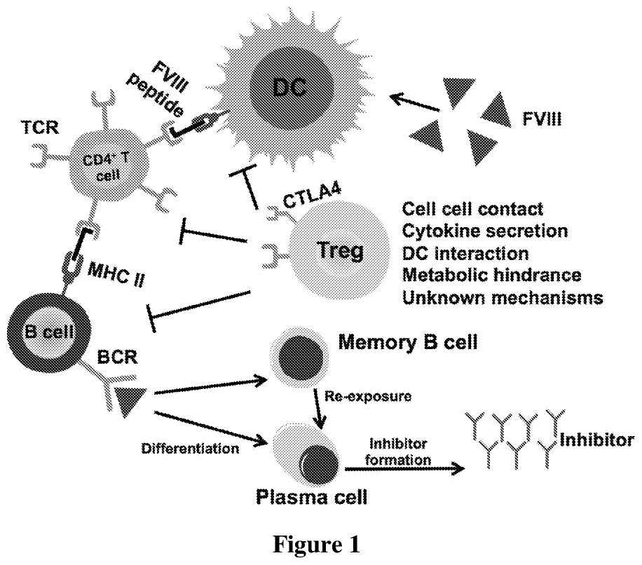

FIG. 2A shows the outline of a human F VIII Chimeric Antigen Receptors (CAR) construct (huF.VIII-CAR) with CD28, 4-1BB and CD3ζ. FIG. 2B shows the mechanism of huF.VIII CAR-Treg activation by soluble and bound F.VIII or by Fc-F.VIII.

FIG. 3 shows a schematic representation of CARs of the first (1G), second (2G), and third generation (3G).

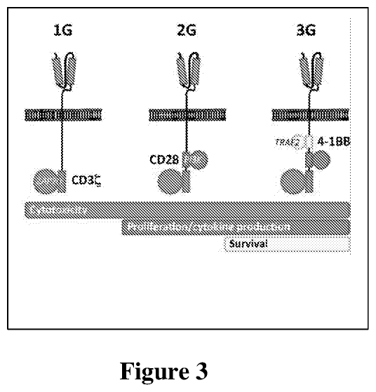

FIG. 4A shows the isolation and in vitro expansion of polyclonal Tregs from splenocytes of BALB/c FoxP3-GFP mice. FIG. 4B shows the expansion of Treg after two rounds of stimulation with anti-CD3/CD28 beads. FIG. 4C shows the percent GFP+ (FoxP3 expressing) cells as a function of days in culture.

FIG. 5A shows that expanded polyclonal Tregs suppress inhibitor formation to F.VIII in mice with pre-existing inhibitor response after receiving a single huF.VIII-CAR Treg injection. FIG. 5B shows that repeat infusions of expanded polyclonal Tregs suppress inhibitor formation against F.VIII in hemophilia A mice receiving factor replacement therapy.

FIG. 6 shows the binding of huF.VIII-CAR-GFP+ Treg cells to Fc-F.VIII (red line) and the binding of GFP+ only Tregs to Fc-F.VIII (black line) followed by staining with A-647 conjugated anti-human IgG.

FIG. 7A shows huF.VIII-CAR expression in Jurkat T cells. FIG. 7B shows a prominent Ca²⁺ flux in huF.VIII-CAR expressing Jurkat cells on incubation with Fc-F.VIII and anti-human Fcγ (orange line). CD3/CD28 bead activated cells (blue and red lines) and mock (GFP only) transduced cells incubated with Fc-F.VIII (light blue line) served as positive and negative controls, respectively.

FIG. 8 shows a schematic of the novel huF.VIII CAR construct of the invention that compared to the construct of FIG. 2A has phenylalanine-to-tyrosine mutations in four of the six CD3ζ ITAMs, i.e., in two out of three pairs of ITAMs, the 4-1BB signaling domain removed and a Myc tag added.

FIG. 9A shows the binding of different concentrations of Fc-F.VIII (10, 5, 2 μg/ml) huF.VIII specific CAR-Tregs (GFP+). FIG. 9B shows detection with AF-647 conjugated anti-Fc using flow cytometry.

FIG. 10 shows CD69 upregulation in huF.VIII CAR Tregs on stimulation with recombinant B-domain deleted (BDD) F.VIII, Fc-F.VIII+anti-Fc, or a non-specific antigen, Fc-F.IX+anti-Fc.

FIG. 11 shows CD4+ conventional T cells retrovirally transduced with huF.VIII CAR labeled with the vital dye CellTraceViolet (CTV) and cultured for 72 h in the present of recombinant BDD-F.VIII, Fc-F.VIII (ELOCTATE)+anti-Fc, Fc-F.IX+anti-Fc, or anti Fc only.

FIG. 12 is a plasmid map for pMYs-IRES-GFP Retroviral Vector, Catalog #RTV-021.

FIG. 13 provides graphs evidencing that in vitro FVIII stimulated CAR Tregs upregulated expression of the transcription factors IRF-4, ROR-γt, and GATA-3, but not Tbet.

FIG. 14 provides a graph evidencing that CAR expressing Tregs stimulated in vitro with FVIII secrete cytokines IL-10, IL-17, but do not secrete IL-2, IL-6 or IFN-γ. Cytokines were tested by ELISA after 48 hrs of CAR-Treg culture with FVIII or an irrelevant antigen, FIX.

FIG. 15 provides a graph evidencing that adoptive transfer of 1 million FVIII CAR Tregs into BALB/c hemophilia A mice followed by weekly IV injections of FVIII for 4 weeks resulted in suppression of functional inhibitor formation as measured by Bethesda assay. Control mice that received FVIII injections only developed high inhibitors (˜100 BU/ml).

### BRIEF DESCRIPTION OF SEQUENCES

SEQ ID NO:1 is the sequence of the CD3ζ domain of a CD19-CAR.

SEQ ID NO:2 is the sequence of a CD3ζ domain of the invention comprising six, i.e., three pairs of tyrosine-containing functional ITAMs.

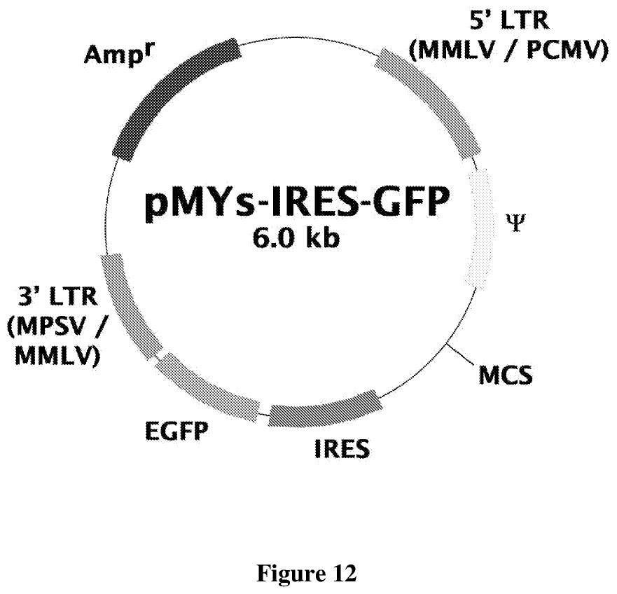

SEQ ID NO:3 is the sequence of a plasmid (pMYs-IRES-GFP Retroviral Vector, Catalog #RTV-021) purchased from Cell Biolabs, Inc. (San Diego, Calif. 92126 USA) used in the subject invention. The plasmid map is illustrated in FIG. 12.

Plasmid Sequence with Key Features
320-614: 5′ LTR
615-1528: psi+ (Extended packaging signal)
1529-1906: Mo-MuLV pol region containing splice acceptor
1918-1993: MCS
2021-2573: IRES
2588-3307: GFP
3373-3963: MuLv 3′ LTR
5016-5878: Ampicillin Resistance

### Definitions

According to the teachings herein, subjects that may be treated with composition and method embodiments include human and non-human animals. The term non-human animal, as used herein, includes, but are not limited to, apes, chimpanzees, orangutans, monkeys; and domesticated animals such as dogs, cats, horses, cattle, pigs, sheep, goats, chickens, mice, rats, guinea pigs, and hamsters. The subject invention provides methods having both human and veterinary utility. Non-human mammalian species which benefit from the disclosed methods include, and are not limited to, apes, chimpanzees, orangutans, monkeys; domesticated animals (pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, giant pandas, hyena, seals, sea lions, and elephant seals.

The term “regulatory T cell” (“Treg”), as used herein, refers to a T cell that is CD4+, CD25+, FoxP3+, secretes regulatory cytokines including, but not limited to, TGFβ and IL-10, and performs immune suppressive functions.

The term “antibody,” as used herein, refers to an immunoglobulin molecule which specifically binds to an antigen and which can be an intact immunoglobulin derived from natural sources or from recombinant sources and can be an immunoreactive portion of an intact immunoglobulin. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, in: Antibodies: A Laboratory Manual, Cold Spring Harbor, N.Y.; Huston et al., 1988; Bird et al., 1988).

The term “antigen,” as used herein is defined as a molecule that provokes an immune response, which immune response may involve antibody production, the activation of specific immunologically-competent cells, or both. The skilled artisan will understand that any macromolecule, including virtually all proteins or peptides, can serve as antigens.

The term “effective amount,” as used herein, refers to an amount that is capable of treating or ameliorating a disease or condition or otherwise capable of producing an intended therapeutic effect.

The terms “about” or “approximately” means within an acceptable error range for the particular value as determined by one of ordinary skill in the art, which will depend in part on how the value is measured or determined, i.e., the limitations of the measurement system. In the context of compositions containing amounts of ingredients where the terms “about” or “approximately” are used, these compositions contain the stated amount of the ingredient with a variation (error range) of 0-10% around the value (X±10%). In a numerical context, such as a contiguous span of nucleotides or amino acids, the terms “about” or “approximately” mean that the span can contain 0-10% more or fewer nucleotides or amino acids (rounded to the closest whole number). For example, a span of about 100 consecutive nucleotides or amino acids can span between 90 and 110 consecutive nucleotides or amino acids.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

The term “therapeutically effective amount,” as used herein, refers to the amount of the subject compound that is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “encoding,” as used herein, refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term “expression,” as used herein is defined as the transcription and/or translation of a particular nucleotide sequence driven by its promoter.

As used herein, the terms “co-administered, “co-administering,” or “concurrent administration”, when used, for example with respect to administration of an exemplary agent (e.g. CAR expressing Tregs or composition comprising CAR expressing Tregs) with another agent (e.g. FVIII) refers to administration of the exemplary agent and the other agent such that both can simultaneously achieve a physiological effect. The agent and other agent, however, need not be administered together. In certain embodiments, administration of one agent can precede or succeed administration of the other or be administered simultaneously, however, such co-administering typically results in both agents being simultaneously present in the body (e.g. in the plasma) of the subject.

The term “lentivirus,” as used herein, refers to a genus of the Retroviridae family, which is unique among the retroviruses in being able to infect non-dividing cells and delivering a significant amount of genetic information into the DNA of the host cell. HIV, S1V, and FIV are all examples of lentiviruses.

The term “inducing tolerance to huF.VIII protein therapy” as used herein refers to reduction of inhibitor formation.

The term “transfected” or “transformed” or “transduced,” as used herein, refers to a process by which exogenous nucleic acid is transferred or introduced into the host cell.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DETAILED DISCLOSURE

Provided are materials and methods to generate human F.VIII specific chimeric antigen receptors (huF.VIII-CARs) expressed in autologous CD4+CD25+Foxp3+ Treg cells to suppress inhibitor formation in hemophilia. The CARs of the subject invention combine the antibody specificity with T cell regulatory function without the need for MHC restriction.

Polyclonal expanded Tregs have been previously identified as an effective prophylactic therapy to prevent inhibitor formation in hemophilia A. However, when tested in animal models with pre-existing inhibitors, this therapy was only partially effective. The antigen-specific CAR expressing Tregs of the subject invention provide improved effectiveness of suppression of inhibitor formation and a reduction in side effects observed with polyclonal Treg-based therapies. Therefore, antigen-specific CAR expressing Tregs of the subject invention can be used at lower numbers to induce tolerance in subjects suffering from hemophilia and inhibitor formation.

Because CARs enable direct antigen binding without the need for presentation by a specific MHC, the cellular therapy of the subject invention using Tregs expressing antigen-specific CARs is an attractive candidate for wide applicability to diverse populations of patients. The antigen-specific CAR expressing Tregs of the subject invention target both B and T cells involved in inhibitor formation through direct and indirect mechanisms.

The subject invention further provides optimized techniques for the isolation and ex vivo expansion of CD4+CD25+FoxP3+ cells and means for adjusting therapeutic doses, number of doses and dose schedule to achieve a therapeutically effective amount of antigen-specific CAR expressing Tregs in the treatment of hemophilia A. Advantageously, the antigen-specific CAR expressing Tregs of the subject invention allow the use of reduced numbers of Tregs in clinical protocols compared to the relatively large numbers of Tregs that are required in current adoptive Treg-based therapies using polyclonal Tregs.

### Overview

Regulatory T cells (Tregs) are a subset of CD4+ helper T cells that are typically defined as CD4+ CD25+ FoxP3+ lymphocytes, and are regarded as one of the most important regulators of peripheral tolerance. Through a variety of mechanisms, including cytokine release and contact-dependent interactions, they can prevent immune responses in an antigen-specific manner.

Because of their great potential as modulators of immunity, Tregs represent an ideal therapeutic tool (Sarkar et al., 2014). Tregs have several advantages compared to other immune modulatory drugs, including a natural immune regulatory ability, avoidance of severe side effects and global immune suppression typically associated with conventional drugs, reduced risk of long-term damage to the immune system, and potential for a lasting tolerogenic response.

Treg based therapies utilizing adoptive Treg transfusion of freshly isolated or ex vivo expanded FoxP3+ or Tr1 Treg subsets have been translated into clinical practice for preventing graft-versus-host disease in patients undergoing allogeneic hematopoietic stem cell transplantation, inhibiting rejection in solid organ transplantation, inflammatory bowel disease, treating hematological cancers, controlling autoimmunity in patients with type 1 diabetes, among others (Perdigoto et al., 2016) (see also, e.g., http://clinicaltrials.gov/NCT01210664, NCT017955 73, NCT01624077, NCT00602693).

Adoptive therapy with Tregs has facilitated preclinical and translational studies. It has also been shown that ex vivo expanded Treg are functionally superior to freshly isolated Treg (Sarkar et al., 2014). Although hard to optimize, efforts to manufacture Tregs cells have led to good-manufacturing-practice (GMP)-grade protocols to isolate and expand human Tregs ex vivo without losing their suppressive function. Polyclonal or antigen specific Treg can be ex vivo expanded by more than 1000-fold (Perdigoto et al., 2016).

Chimeric antigen receptor (CAR)-based approaches have been used in immunotherapies to combine the specificity of a monoclonal antibody with the proliferative and cytotoxic abilities of an activated T cell. In CAR technology, antigen receptor and co-stimulatory molecule signaling is complexed with antibody-based antigen recognition, bypassing the need for HLA restriction or the requirement for antigen presenting cells. Three generations of CARs have been developed with different combinations of signaling domains, with 2nd and 3rd generation CARs showing the greatest efficacy.

For example, CAR technology has been successfully used in cancer immunotherapy. Genetic modifications of autologous CD4+ or CD8+ T cells engineered to recognize and kill cells through a CAR have been shown to be highly effective at eradicating B cell leukemias and lymphomas that are resistant to standard therapies in cancer patients. CARs have been successfully employed in clinical trials of modified T cells in patients with relapsed and refractory B-cell lymphoma, B-cell leukemias, including chronic lymphocytic leukemia (CLL) and acute lymphoblastic leukemia (ALL) (Carpenito et al., 2009; Brentjens et al., 2011; Porter et al., 2011; Kochenderfer et al., 2012; Maude et al., 2014). Autologous CD8+ T cells engineered to express a CAR molecule have shown potent, non-cross reactive activity, with progression to stable disease, partial and complete remissions (Carpenito et al., 2009; Brentjens et al., 2011; Porter et al., 2011; Kochenderfer et al., 2012; Maude et al., 2014; Ardouin et al., 1999; Kochenderfer et al., 2010). Rapid trafficking to the site of tumor, proliferation in vivo, persistence of adoptively transferred cells, and in some cases, conversion to memory CAR T cells has been observed.

However, serious adverse events including B cell aplasia, tumor lysis syndrome and cytokine release syndrome have also been reported (Carpenito et al., 2009; Brentjens et al., 2011; Porter et al., 2011; Kochenderfer et al., 2012; Maude et al., 2014). For example, CARs specific for CD19 to deplete B cells can raise concerns of general immunosuppression, leaving the body vulnerable to opportunistic infections. Other concerns include continuous activation that can cause uncontrollable release of cytokines or off-target toxicity.

CARs can comprise different signaling domains. For example, all CARs comprise the primary CD3ζ signaling domain. Second and third generation CARs comprise additional co-stimulatory signaling domains such as CD28 and 4-1BB. Such CARs have been shown to increase persistence in studies of B-ALL (Carpenito et al., 2009).

CD3ζ domains comprise immune-receptor tyrosine-based motifs (ITAMs) involved in downstream signaling. However, inactivation of some select ITAMs in the CD3ζ signaling domain of T cell receptors has been shown to not impair TCR signaling (Ardouin et al., 1999). Because excessive signaling through CARs expressed in conventional T cells has been linked to T cell apoptosis and reduced functionality of the CAR expressing T cells in vivo, CARs with CD3ζ domains having four of six ITAMs, i.e., two out of three pairs of ITAMs inactivated or crippled by tyrosine-to-phenylalanine mutation have been generated and reduced T cell apoptosis and improved in vivo T cell function was observed (Kochenderfer et al., 2010).

Conventional T cells transduced with CARs and exposed to CAR ligands execute multiple key therapeutic functions upon antigen ligand engagement including, but not limited to, production of antitumor cytokines and killing of target cells.

CAR Tregs are an innovative concept. CAR Tregs have the potential to suppress CAR ligand specific effector T cells and other immune cell types by mechanisms such as interfering with T cell metabolism or by interacting with dendritic cells to convert them into a more regulatory phenotype. However, no ligand specific CAR Tregs for suppression of antibody formation in subjects treated with therapeutic proteins have been devised and the feasibility of ligand specific CAR Tregs for treating inhibitor formation in hemophilia A is heretofore unknown.

### DETAILED DESCRIPTION OF EMBODIMENTS

The CARs of the subject invention comprise antibody-derived domains for interaction with a target antigen and signaling domains for induction of intracellular signaling in a CAR expressing cell. In some embodiments, the CARs comprise an antibody-derived domain that is a single chain variable fragment (scFv). In preferred embodiments, the scFv is derived from an antibody of a human subject that has developed anti-F.VIII antibodies (inhibitors) following F.VIII protein therapy. In some embodiments, the scFv is derived from an antibody of the IgG isotype. In further embodiments, the human IgG antibody is of the subclass IgG1, IgG2, IgG3, or IgG4. In other embodiments, the scFv is derived from an antibody of an IgM isotype. In yet other embodiments, the scFv is derived from an antibody of an IgA isotype. In further embodiments, the scFv is derived from an antibody of an IgE isotype. In yet further embodiments, the scFv is derived from an antibody of an IgD isotype. In many embodiments, the scFv comprises a light chain and a heavy chain portion.

In preferred embodiments, the human antibody from which the scFv is derived is a human antibody produced in a B cell originating from a human subject with inhibitor formation following F.VIII protein therapy. In some embodiments, the human antibody from which the scFv is derived is a human antibody produced in a B cell line. In some embodiments, the B cell line is generated by transformation of a B cell derived from a human subject with inhibitor formation following F.VIII protein therapy with a virus. The virus used for transformation can include, but is not limited to, Epstein Barr Virus (EBV), SV40 virus, Marek's Disease Virus (MDV), and an Abelson Murine Leukemia Virus (Ab-MLV).

In some embodiments, the antibody from which the scFv of the CAR is derived is directed against an epitope present anywhere in the target antigen. The antibody from which the scFv of the CAR is derived can be directed against an epitope of a low of about 5 amino acids to a high of about 20 amino acids. For example, the antibody can be directed against an epitope of about 6 amino acids to about 19 amino acids; of about 7 amino acids to about 18 amino acids; of about 8 amino acids to about 17 amino acids; of about 9 amino acids to about 16 amino acids; of about 10 amino acids to about 15 amino acids; of about 11 amino acids to about 14 amino acids; or of about 12 amino acids to about 13 amino acids.

In other embodiments, the antibody from which the scFv of the CAR is derived can be directed against a conformational epitope present in the target antigen. The antibody from which the scFv of the CAR is derived can be directed against a conformational epitope present in a region of the antigen comprising a low of about 21 amino acids to a high of about 500 amino acids. For example, the antibody can be directed to a conformational epitope present in about 22 to about 475 amino acids; about 25 to about 450 amino acids; about 30 to about 425 amino acids; about 40 to about 400 amino acids; about 50 to about 375 amino acids; about 60 to about 350 amino acids; about 70 to about 300 amino acids; about 80 to about 250; about 90 to about 200 amino acids; about 100 to about 150 amino acids; or about 110 to about 125 amino acids. In a preferred embodiment, the antibody from which the scFv of the CAR is derived can be directed against a conformational epitope present in about 100 to about 300 amino acids. In a more preferred embodiment, the antibody from which the scFv of the CAR is derived can be directed against a conformational epitope present in about 125 to about 250 amino acids. In a most preferred embodiment, the antibody from which the scFv of the CAR is derived can be directed against a conformational epitope present in 207 amino acids of the antigen.

In a further preferred embodiment, the antibody from which the scFv of the CAR is derived is specific for a F.VIII protein. In a more preferred embodiment, the antibody from which the scFv of the CAR is derived is specific for a human F VIII protein. However, in other embodiments, the antibody from which the scFv of the CAR is derived is specific for a non-human antigen including, but not limited to, non-human F VIII antigens. In more preferred embodiments, the antibody from which the scFv of the CAR is derived is specific for residues of the C1 domain and the C2 domain of the human F.VIII protein. In a most preferred embodiment, the antibody from which the scFv of the CAR is derived is specific for residues 2125 to 2332 of the human F VIII protein.

In many embodiments, the CAR of the subject invention comprises a CD3ζ signaling domain. In some embodiments, the CAR of the subject invention comprises a CD28 signaling domain. In some embodiments, the CAR of the subject invention comprises a 4-1BB signaling domain. In some embodiments, the CAR of the subject invention comprises a CD3ζ and a 4-1BB signaling domain. In further embodiments, the CAR of the subject invention comprises a CD28 and a 4-1BB signaling domain. In yet further embodiments, the CAR of the subject invention comprises a CD3ζ, a CD28 and a 4-1BB signaling domain. In preferred embodiments, the CAR of the subject invention comprises a CD3ζ and a CD28 signaling domain.

The CD3ζ signaling domain of the CAR of the subject invention comprises at least one immuno-receptor tyrosine-based activation motif (ITAM). An ITAM is known in the art to comprise a YXXL/I sequence, wherein X corresponds to a variable residue. ITAMs are known to be generally separated by 6-8 variable amino acids, wherein the variable amino acid can be an amino acid, including, but not limited to, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiment, the CD3ζ signaling domain of the CAR of the subject invention comprises two ITAMs, e.g., a pair of ITAMs. In some embodiments, the CD3ζ signaling domain of the CAR of the subject invention comprises three ITAMs. In some embodiments, the CD3ζ signaling domain of the CAR of the subject invention comprises four ITAMs, e.g., two pairs of ITAMs. In some embodiments, the CD3ζ signaling domain of the CAR of the subject invention comprises five ITAMs. In some embodiments, the CD3ζ signaling domain of the CAR of the subject invention comprises six ITAMs, i.e., three pairs of ITAMs.

The ITAMs of the CD3ζ of the CAR of the subject invention can be crippled. ITAM crippling comprises a substitution of at least one tyrosine residue of an ITAM with a non-tyrosine amino acid, wherein the non-tyrosine amino acid can be phenylalanine or tryptophan. For example, one, two, three, four, five, or six ITAMs of the CAR can be crippled. Furthermore, the crippling of ITAMs one, two, five, and six of a CD3ζ in the context of a CAR expressed on a conventional T cell can decrease apoptosis of said T cell and enhance the efficiency of CAR-conventional T cell therapy. In some embodiments, at least one tyrosine residue of the ITAM of the CD3ζ of the CAR of the subject invention is substituted with a phenylalanine.

Advantageously, it has been discovered that reversal of crippled ITAMs back to tyrosines in CARs expressed on Tregs significantly improves functionality of Tregs expressing said CARs. Therefore, in some embodiments, the CARs of the subject invention comprise one tyrosine in the six ITAMs of the CD3ζ signaling domain. In some embodiments, the CARs of the subject invention comprise two tyrosines in the six ITAMs of the CD3ζ signaling domain. In some embodiments, the CARs of the subject invention comprise three tyrosines in the six ITAMs of the CD3ζ signaling domain. In preferred embodiments, the CARs of the subject invention comprise four tyrosines in the six ITAMs of the CD3ζ signaling domain. In more preferred embodiments, the CARs of the subject invention comprise five tyrosines in the six ITAMs of the CD3ζ signaling domain. In most preferred embodiments, the CARs of the subject invention comprise six tyrosines in the six ITAMs of the CD3ζ signaling domain.

Advantageously, the presence of six tyrosines in the six ITAMs of the CD3ζ signaling domain of a CAR of the subject invention enables optimal activation of Tregs expressing said CAR and surprisingly superior in vitro and in vivo performance of the Treg expressing said CAR leading to superior suppression of inhibitor formation in animals treated with Tregs expressing said CAR compared to animals treated with Tregs expressing a CAR not having six tyrosines in the six ITAMs of the CD3ζ signaling domain.

In a preferred embodiment, the CAR of the subject invention comprises a scFv specific for F.VIII comprising a light and a chain region, a myc tag, a CD28 signaling domain and a CD3ζ signaling domain comprising six tyrosines in the six ITAMs. The subject invention also provides nucleic acids encoding a CAR of the subject invention. In preferred embodiments, the nucleic acid encodes a CAR that is specific for human F.VIII. In further preferred embodiments, the nucleic acid is derived from a B cell originating from a human subject with inhibitor formation following F.VIII protein therapy. In other embodiments, the nucleic acid is derived from a B cell line generated by transformation of a B cell derived from a human subject with a virus.

In some embodiments, the nucleic acid encoding a CAR of the subject invention comprises a nucleic acid sequence encoding a single chain variable fragment (scFv). In preferred embodiments, the nucleic acid encoding the scFv is derived from a nucleic acid encoding an antibody of a human subject that has developed anti-F.VIII antibodies (inhibitors) following F.VIII protein therapy. In some embodiments, the nucleic acid encoding the scFv is derived from a nucleic acid encoding an antibody of the IgG isotype. In further embodiments, the nucleic acid encoding the scFv is derived from a nucleic acid encoding an antibody of the subclass IgG1, IgG2, IgG3, or IgG4. In other embodiments, the nucleic acid encoding the scFv is derived from a nucleic acid encoding an antibody of an IgM isotype. In yet other embodiments, the nucleic acid encoding the scFv is derived from a nucleic acid encoding an antibody of an IgA isotype. In further embodiments, the nucleic acid encoding the scFv is derived from a nucleic acid encoding an antibody of an IgE isotype. In yet further embodiments, the nucleic acid encoding the scFv is derived from a nucleic acid encoding an antibody of an IgD isotype. In many embodiments, the nucleic acid encoding the scFv comprises a nucleic acid encoding a light chain and a heavy chain portion.

In preferred embodiments, the nucleic acid encoding the scFv is derived from a nucleic acid encoding an antibody that is specific for a F.VIII protein. In a more preferred embodiment, the nucleic acid encoding the scFv is derived from a nucleic acid encoding an antibody that is specific for a human F.VIII protein. However, nucleic acids derived from antibodies that are specific for non-human antigens including, but not limited to, non-human F.VIII antigens are also included. In more preferred embodiments, the nucleic acids encoding the scFv is derived from a nucleic acid encoding an antibody that is specific for residues of the C1 domain and the C2 domain of the human F VIII protein. In the most preferred embodiment, the nucleic acid encoding the scFv is derived from a nucleic acid encoding an antibody that is specific for residues 2125 to 2332 of the human F VIII protein.

In many embodiments, the nucleic acids encoding CARs of the subject invention comprise a nucleic acid encoding a CD3ζ signaling domain. In some embodiments, the nucleic acids encoding CARs of the subject invention comprise a nucleic acid encoding a CD28 signaling domain. In some embodiments, the nucleic acids encoding CARs of the subject invention comprise a nucleic acid encoding a 4-1BB signaling domain. In further embodiments, the nucleic acids encoding CARs of the subject invention comprise nucleic acids encoding a CD3ζ signaling domain and a 4-1BB signaling domain. In yet further embodiments, the nucleic acids encoding CARs of the subject invention comprise nucleic acids encoding a CD28 signaling domain and a 4-1BB signaling domain.

In preferred embodiments, the nucleic acids encoding CARs of the subject invention comprise nucleic acids encoding a CD3ζ signaling domain and a CD28 signaling domain. In some preferred embodiments, the nucleic acids encoding the CD3ζ signaling domain of the CARs of the subject invention comprise nucleic acid sequences encoding at least one tyosine within the six ITAMs of the CD3ζ signaling domain. In some preferred embodiments, the nucleic acids encoding the CD3ζ signaling domain of the CARs of the subject invention comprise nucleic acid sequences encoding two tyrosines within the six ITAMs of the CD3 signaling domain. In some preferred embodiments, the nucleic acids encoding the CD3 signaling domain of the CARs of the subject invention comprise nucleic acid sequences encoding three tyrosines within the six ITAMs of the CD3ζ signaling domain. In some preferred embodiments, the nucleic acids encoding the CD3ζ signaling domain of the CARs of the subject invention comprise nucleic acid sequences encoding four tyrosines within the six ITAMs of the CD3ζ signaling domain. In more preferred embodiments, the nucleic acids encoding the CD3ζ signaling domain of the CARs of the subject invention comprise nucleic acid sequences encoding five tyrosines within the six, i.e., three pairs of ITAMs of the CD3 signaling domain. In most preferred embodiments, the nucleic acids encoding the CD3 signaling domain of the CARs of the subject invention comprise nucleic acid sequences encoding six tyrosines within the six ITAMs of the CD3ζ signaling domain.

In preferred embodiments of the subject invention, CD4+CD25+FoxP3+ Tregs are provided that have been transduced with antigen-specific CARs of the subject invention to combine specific immunosuppression with an improved safety profile. Upon antigen ligand engagement, conventional CAR T cells (which include CD4+ and CD8+ T cells) execute multiple key therapeutic functions, including production of antitumor cytokines and killing of target cells. CAR-transduced Tregs of the subject invention execute upon antigen ligand engagement suppressor functions that are specific to the antigen and reduce formation of antibodies against the antigen, or inhibitors, in a subject.

Methods of T cell transduction including Treg cell transduction are known in the art. For example, efficient Treg cell transduction is achieved using retroviral and lentiviral vectors available in the art. Any and all such methods and vectors are included herein. Advantageously, Tregs of the subject invention transduced with human F.VIII specific CARs of the subject invention proliferate when contacted with human F VIII causing contact dependent or cytokine dependent suppression of inhibitor forming plasma cells. Surprisingly, activation and functionality of F.VIII-specific CAR expressing Tregs of the subject invention are significantly increased when contacted with F.VIII-Fc fusion proteins. Even further improvement in activation and functionality of F.VIII-specific CAR expressing Tregs of the subject invention is achieved by binding F.VIII-Fc fusion proteins with cross-linking antibodies.

The F.VIII-specific CAR expressing Tregs of the subject invention have surprisingly superior functionality when activated by inhibitors bound to B cells or inhibitors present in circulating immune complexes with F.VIII in subjects in vivo. Further, F.VIII-specific CAR expressing Tregs of the subject invention suppress F.VIII-specific effector T cells and other immune cell types by mechanisms including, but not limited to, interference with T cell metabolism and interaction with dendritic cells to convert them into a regulatory phenotype.

Also provided herein are animal models of hemophilia that are useful in the determination of therapeutic efficiency of Treg cell-based therapies to suppress inhibitor formation.

In one example, inhibitors are generated in male BALB/c F8e16^{-/-} mice by 4-8 weekly intravenous administrations of recombinant human F.VIII. Initial anti-F.VIII IgG1 production and inhibitor formation is monitored by ELISA measurement and Bethesda assay. Mice are injected with either expanded polyclonal Treg (group 1), human F.VIII-specific CAR expressing Tregs (group 2), mock GFP transduced Tregs (group 3), or nothing (group 4), at a starting dose of 1×10⁶ Treg/mouse. In some embodiments, the dose of Tregs injected is increased by increasing number of dose. In other embodiments, the dose of Tregs injected is increased by increasing the Treg cells per dose injected. Mice continue to receive 4 weekly F.VIII or Fc-F.VIII injections. Using this model, human F.VIII-specific CAR expressing Tregs of the subject invention have the capability to suppress and reverse inhibitor formation.

Advantageously, human F.VIII-specific CAR expressing Tregs of the subject invention persist in vivo for an extended period of time with presence of Tregs of the subject invention in blood, spleen, liver, and peripheral lymph nodes. The term “subject,” as used herein, describes an organism, including mammals such as primates, to which treatment with the compositions according to the subject invention can be provided.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain embodiments of the subject invention, any number of T cell lines available in the art, may be used. In preferred embodiments, Tregs are isolated and purified from blood or bone marrow of a subject into which the Treg-enriched composition is subsequently introduced.

Alternatively, Tregs may be obtained from a donor distinct from the subject. In certain embodiments, T cells may be obtained from a unit of blood collected from a subject using any number of techniques known to the skilled artisan such as, e.g., FICOLL separation. For example, antibodies that recognize the species-specific varieties of CD4, CD25, CD45RA, CD127 and other markers can be used to enrich for or isolate Treg cells from a human.

In particular embodiments, Tregs are enriched from a population of cells using reagents that bind cell surface markers specific for Tregs and Tregs are separated using cell sorting assays such as fluorescence-activated cell sorting (FACS), solid-phase magnetic beads, etc., as known in the art. In some embodiments, combinations of methods to sort cells can be used, e.g., magnetic selection, followed by FACS.

Whether prior to or after genetic modification of the Tregs to express a desirable CAR, the Tregs can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; or 6,867,041, which are incorporated herein in their entirety.

### Methods and Materials

All patents, patent applications, provisional applications, and publications referred to or cited herein are incorporated by reference in their entirety, including all figures and tables, to the extent they are not inconsistent with the explicit teachings of this specification.

The following examples illustrate materials and procedures for making and practicing the invention. These examples should not be construed as limiting. All percentages are by weight and all solvent mixture proportions are by volume unless otherwise noted. It will be apparent to those skilled in the art that the example involves use of materials and reagents that are commercially available from known sources, e.g., chemical supply houses, so no details are given respecting them.

### Example 1

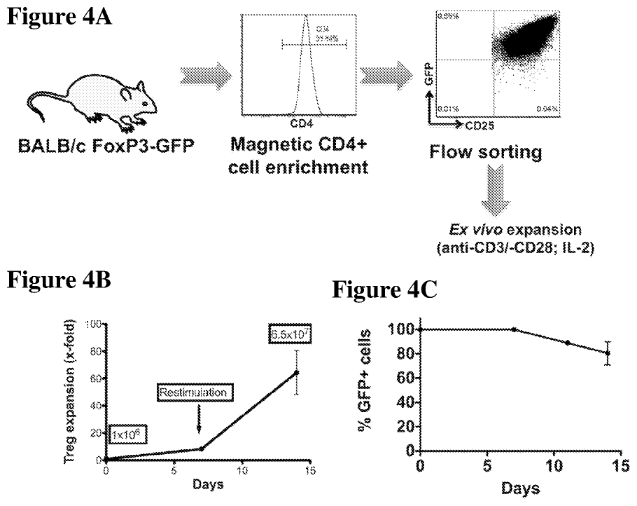

In vitro expansion of murine Tregs was successfully optimized. GFP+ cells were purified (>98% purity) from spleens of FoxP3-GFP reporter mice using flow sorting. Sorted cells were stimulated in culture using anti-CD3/CD28 beads in the presence of high levels of IL-2 (2000 U/ml). About 20- to 100-fold expansions within 14 days were routinely accomplished (FIGS. 4A-4B). Expanded Tregs were ˜80% FoxP3 positive (FIG. 4C).

### Example 2

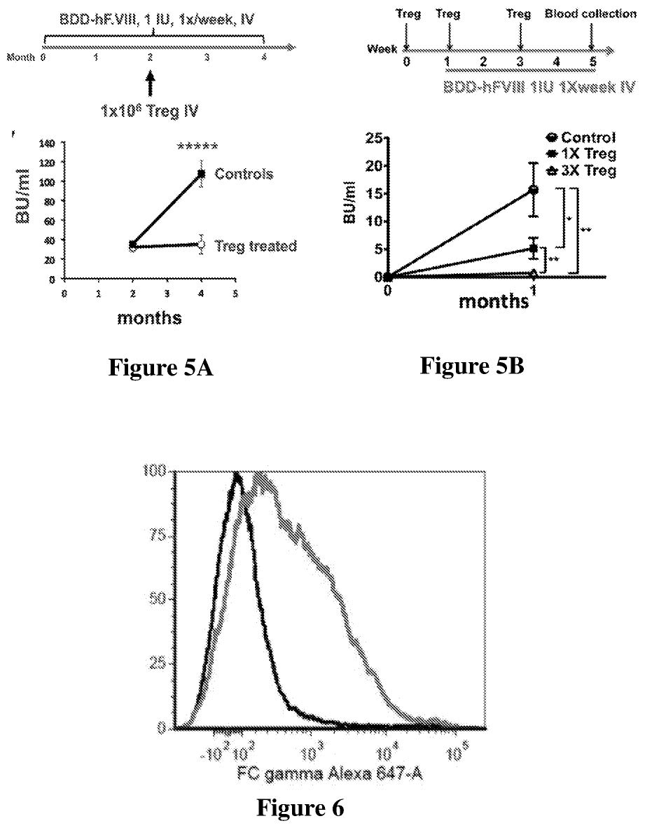

BALB/c-derived Tregs expanded in vitro were injected (1×10⁶ Tregs/mouse) into hemophilia A mice with exon 16 deletion (BALB/c F8e16^{-/-}) with established inhibitors. Treg therapy controlled antibody titers in these mice despite continued F.VIII administration suggested that polyclonal Tregs aid in ITI (FIG. 5A).

Three repeat infusions of expanded 1×10⁶ Tregs were able to suppress inhibitors more effectively than a single dose (FIG. 5B).

### Example 3

A 3^{rd} generation CAR specific for human F VIII was generated in a retroviral system (pMys-IRESGFP, see FIG. 12 and SEQ ID NO: 3). To this end, an EBV transformed B cell line (BO2C11; originally developed by Saint-Remy and colleagues, kindly provided by Dr. David Scott), which produces IgG4 against residues 2125-2332 of huF.VIII corresponding to the carboxy-terminal end of C1 and the complete highly immunogenic C2 domain was used.

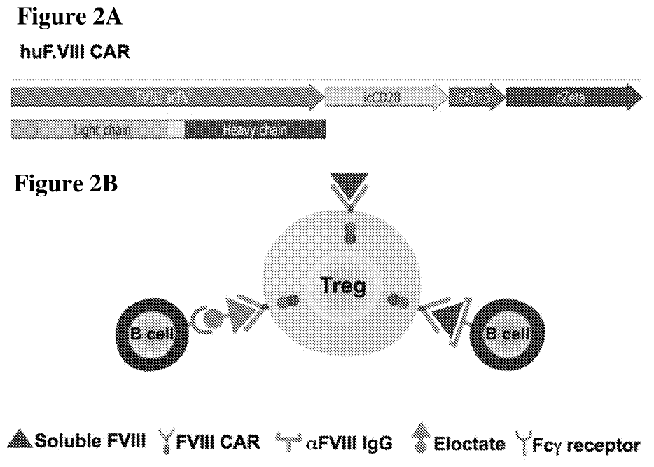

The single chain variable fragment (scFv) was cloned and fused to a 3^{rd} generation CAR construct expressing CD3ζ, CD28 and 4-1BB signaling molecules (received from Dr. Angelica Loskog, Uppsala University) (FIG. 2A). About 25-50% transduction of CD3/28 bead-activated CD4+CD25+ Tregs was achieved as quantified by GFP expression.

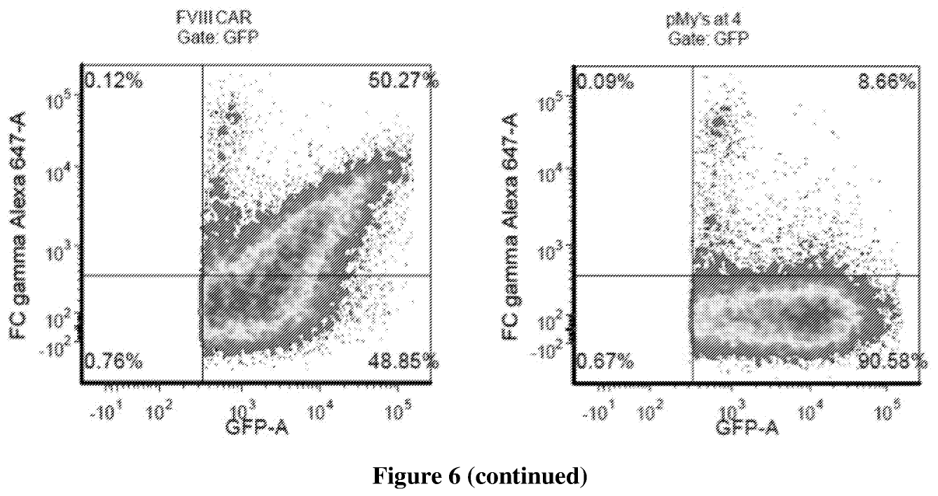

Initially, no activation of huF.VIII CAR-Treg in response to free human F VIII in vitro was observed. However, binding to an Fc fusion F.VIII product (Biogen, Cambridge, Mass.) in the presence of cross-linking antibody (anti-huIgG Fcγ) was demonstrated (FIG. 6).

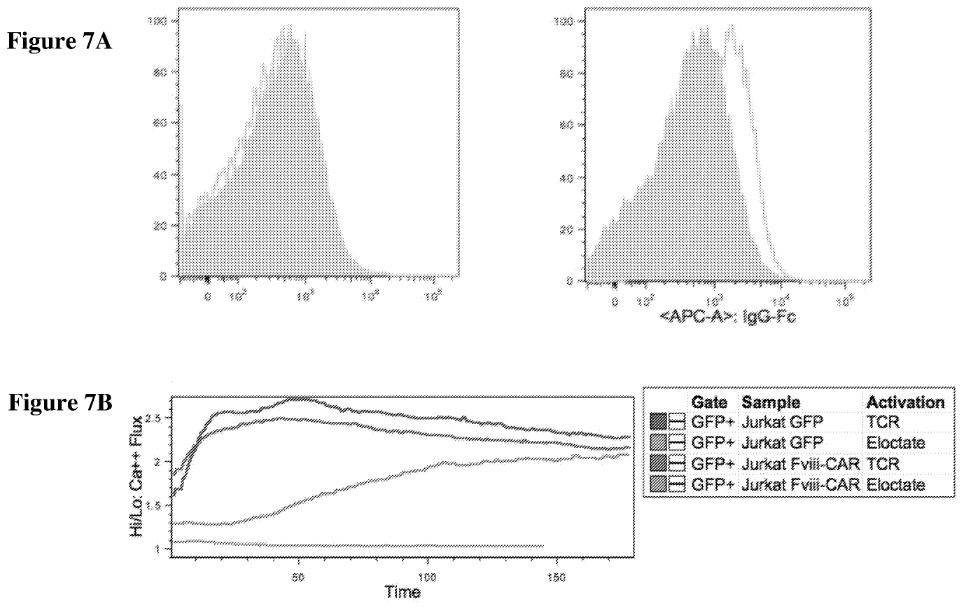

In parallel, a huF.VIII CAR was cloned into the pCNFW.T2A.eGFP lentiviral construct designed to eventually target human Tregs. Binding of human Jurkat T cells only to Fc-F.VIII and not free F.VIII was confirmed (FIG. 7A).

Furthermore, the ability of the CAR construct to be activated upon ligation of the receptor was tested. A rapid and proximal indicator of T cell activation involves the flux of Ca++ upon receptor ligation. A prominent Ca++ flux in huF.VIII-CAR Jurkat cells but not GFP+ mock-transduced control cells following incubation with Fc-F.VIII, and crosslinking with anti-huIgG Fcγ was demonstrated (FIG. 7B).

### Example 4

The ability of huF.VIII CAR-Tregs to suppress T and B cell responses to F.VIII in vitro was tested. Retrovirally transduced huF.VIII CAR Tregs were tested using plate bound Fc-F.VIII and anti-huIgG Fcγ. Antibodies to CD69 and the cell proliferation marker Ki67 were used to determine activation. Human F VIII CAR Tregs were assessed for their ability to suppress antigen-specific CD4+CD25− T cells and/or polyclonal responder T cells. A dual color suppression assay was used to simultaneously monitor effector and responder activity. Responder T cells (labelled with CellTraceViolet, Invitrogen, Carlsbad, Calif.) were plated with effector huF.VIII CAR-Tregs (GFP+) added at varying ratios (10:1, 2:1, 1:1 . . . 0:1 effector: responder). Proliferation was assessed by flow cytometry. Percent suppression was calculated as [1−(prolif. index of Treg+Tresp)/(prolif. index of Tresp alone)]×100%.

### Example 5

Human F VIII specific effector T cells were generated by immunization of strain-matched mice with F.VIII. An in vitro B cell suppression assay employing splenocytes and bone marrow cells from immunized mice (sorted CD19+ B cells or CD138+ plasma cells) was used.

### Example 6

The suppressive activity of CAR Tregs for reversal of inhibitor formation in vivo in animal models of hemophilia was tested. It was observed that Tregs transduced with huF.VIII CAR were poorly activated by free huF.VIII, but showed substantial binding to Fc-F.VIII with cross-linking antibody (FIGS. 6 and 7). This indicated that huF.VIII CAR Treg could be activated in the presence of inhibitors bound to B cells or as circulating immune complexes with FVIII in vivo. A model for reversal studies in mice with existing inhibitors was generated. Inhibitors were induced in cohorts of male BALB/c F8e16^{-/-} mice by 4-8 weekly intravenous administrations of recombinant human F VIII (1 IU per administration). Initial anti-F.VIII IgG1 production (ELISA) and inhibitor formation (Bethesda assay) was monitored by monthly bleeding. Mice were injected with either expanded polyclonal Tregs (group 1), huF.VIII CAR Tregs (group 2), mock GFP transduced Tregs (group 3), or nothing (group 4), at a starting dose of 1×10⁶ Tregs/mouse. Repeat dosing was also performed. Mice continued to receive 4 weekly F.VIII or Fc-F.VIII injections. The ability of huF.VIII CAR Tregs to suppress or reverse inhibitors was tested both by IgG1 ELISA and Bethesda assay.

In separate experiments, persistence and biodistribution of huF.VIII CAR Tregs was monitored in mice after adoptive transfer of CAR Tregs. GFP and ScFv expression was used as a dual marker to detect engrafted Tregs in blood, spleen, liver and peripheral lymph nodes.

### Example 7

Because there is the possibility that strong costimulatory signals (CD28 and 4-1BB signaling chains) may lead to excessive Treg activation and proliferation, resulting in the potential for Treg instability, alternative strategies can be used. For example, 1st and 2^{nd} generation CARs can be tested. Furthermore, because Treg therapies can potentially result in non-specific suppression, it is important to attenuate the adaptive immune response to F.VIII while simultaneously preserving protective immune responses to third party antigens needed for protective immunity. A non-specific antigen like keyhole limpet hemocyanin was administered to mice that received huF.VIII CAR Tregs in order to test for responses to an unrelated antigen.

### Example 8

In initial experiments sub-optimal activation and proliferation by the F.VIII CAR expressing Tregs and conventional T cells was observed. Upon sequencing the original CD19-CAR from which the F.VIII-specific CAR was derived, it was observed that four of the six ITAMs, i.e., two out of three pairs of ITAMs responsible for signaling in the CD3ζ domain had been crippled. This crippling is commonly used with CD19-CARs, which undergo massive proliferation and apoptosis in leukemia models (Ardouin et al., 1999).

The crippling of ITAMs of the CD3ζ domain, however, was not optimal for Treg CARs, which have a different threshold of activation.

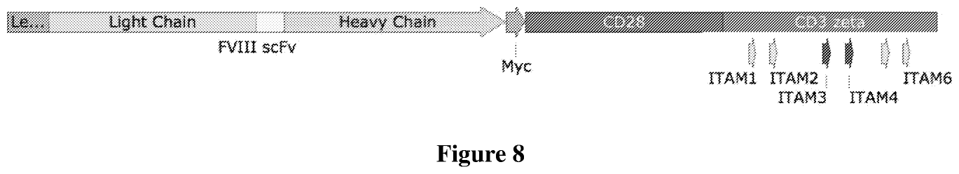

Therefore, the tyrosine-to-phenylalanine mutations in the crippled ITAMs were reversed by site directed phenylalanine-to-tyrosine mutagenesis to render all six, i.e., all three pairs of ITAMs of the CD3ζ domain functional. Further included was a Myc tag for easy detection and the 4-1BB domain was removed (FIG. 8).

The novel CAR construct also contained a F.VIII specific scFv from an EBV transformed B cell line which produced IgG4 directed against residues 2125-2332 of huF.VIII (C1-C2 domains) (FIG. 8). The resulting construct was cloned into the retroviral vector pMys-eGFP.

### Example 9

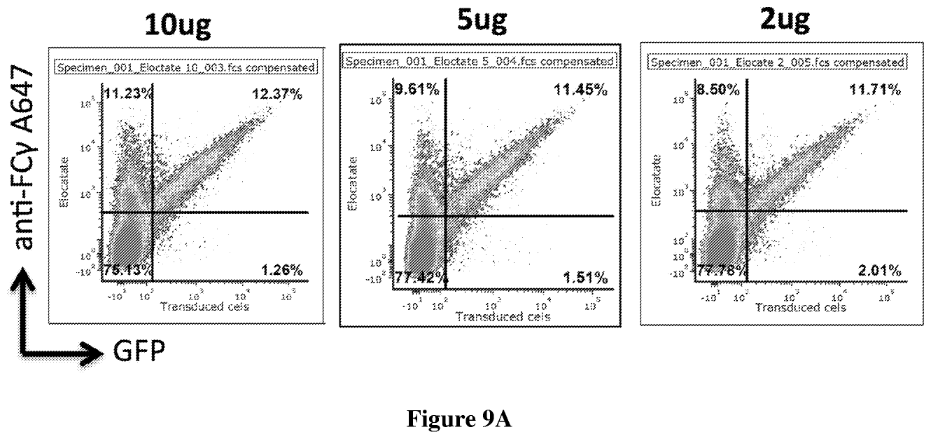

The novel F.VIII CAR was expressed in conventional T cells and Treg cells. Both cell types showed specific binding, activation and proliferation in vitro. Tregs expressing the novel F.VIII CAR showed binding was F.VIII dose-dependent (FIG. 9).

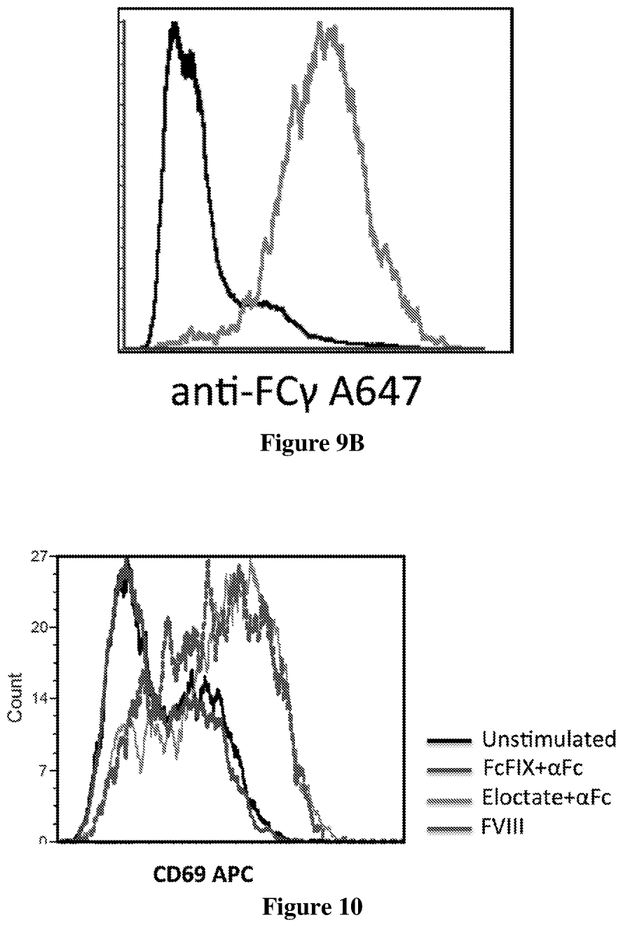

Tregs expressing the novel F.VIII CAR also demonstrated activation as observed by CD69 upregulation on stimulation with Fc-F.VIII (Eloctate)+anti-Fc or recombinant BDD-F.VIII (FIG. 10). No activation was observed on incubation with Fc-F.IX.

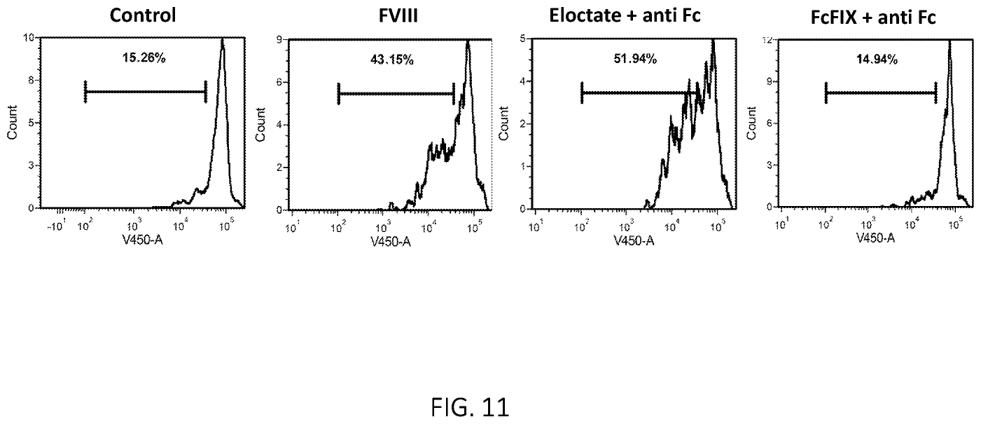

Conventional T cells expressing the novel F.VIII CAR also proliferated in vitro in response to either Fc-F.VIII (Eloctate)+anti-Fc or recombinant BDD-F.VIII (FIG. 11). No proliferation was observed in response to the non-specific protein, Fc-F.IX.

### Example 10

Suppressive activity of F.VIII CAR-Tregs in vivo for the prevention or reversal of inhibitor antibody formation was tested in hemophilia A mice that were given factor replacement therapy with F.VIII. Inhibitors were generated in cohorts of BALB/c F8e16^{-/-} mice (n=6-8/group) by 4-8 weekly intravenous administrations of recombinant human F.VIII (1 IU per administration). Initial anti-F.VIII IgG1 production and inhibitor formation were monitored by monthly bleeding using ELISA and Bethesda Assay, respectively. Mice were injected with either expanded polyclonal Tregs (group 1), F.VIII CAR-Tregs (group 2), mock GFP transduced Tregs (group 3) or nothing (group 4) at a starting dose of 1×10⁶ Tregs/mouse. Repeat dosing was also performed. Mice continued to receive 4 weekly F.VIII injections.

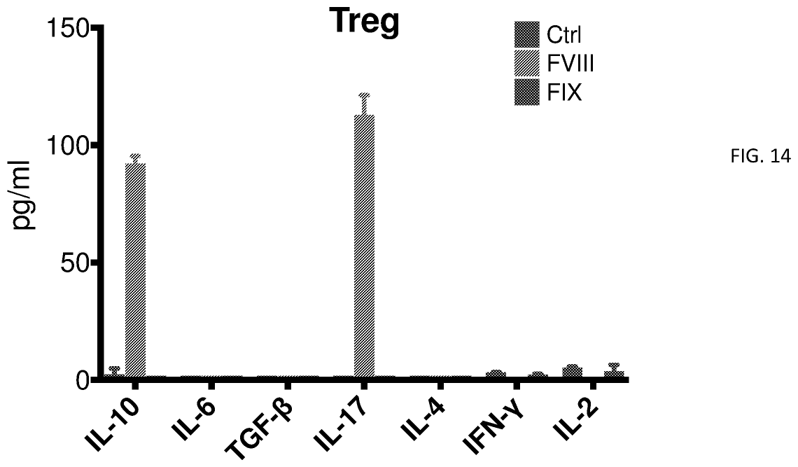

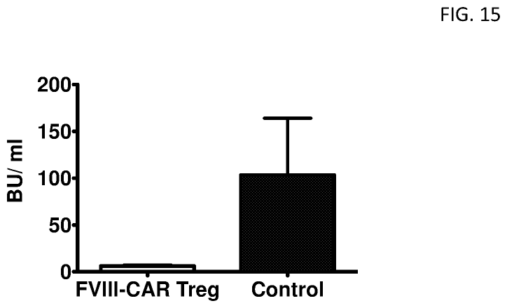

In separate experiments, the persistence and biodistribution of huF.VIII CAR Tregs was monitored in mice at 2, 7, 14, and 30 days after adoptive transfer of huF.VIII CAR Treg. Also, FIG. 15 shows the results of adoptive transfer of 1 million FVIII CAR Tregs into BALB/c hemophilia A mice followed by weekly IV injections of FVIII for 4 weeks. This treatment resulted in suppression of functional inhibitor formation as measured by Bethesda assay. Control mice that received FVIII injections only developed high inhibitors (˜100 BU/ml). Moreover, CAR expressing Tregs stimulated in vitro with FVIII secrete cytokines IL-10, IL-17, but do not secrete IL-2, IL-6 or IFN-γ. Cytokines were tested by ELISA after 48 hrs of CAR-Treg culture with FVIII or an irrelevant antigen, FIX (results shown in FIG. 14).

### Example 11

Concerns about tonic CAR signaling and the presence of endogenous TCR in CAR-transduced cells, which may affect CAR T cell potency were evaluated. By directing a F.VIII-specific CAR to the TCR α constant (TRAC) locus uniform CAR expression in human peripheral blood T cells and enhanced T cell potency can be achieved. In fact, edited cells vastly outperform conventionally generated CAR T cells. Targeting the CAR to the TRAC locus averts tonic CAR signaling and establishes effective internalization and re-expression of the CAR following single or repeated exposure to antigen, delaying T cell exhaustion. Furthermore, CRISPR-Cas can be used to insert the F.VIII CAR construct into the endogenous TCR locus of Tregs to improve stability and persistence of CAR Tregs in a hemophilia model.

### Example 12

### FVIII Stimulates Transcription Factors

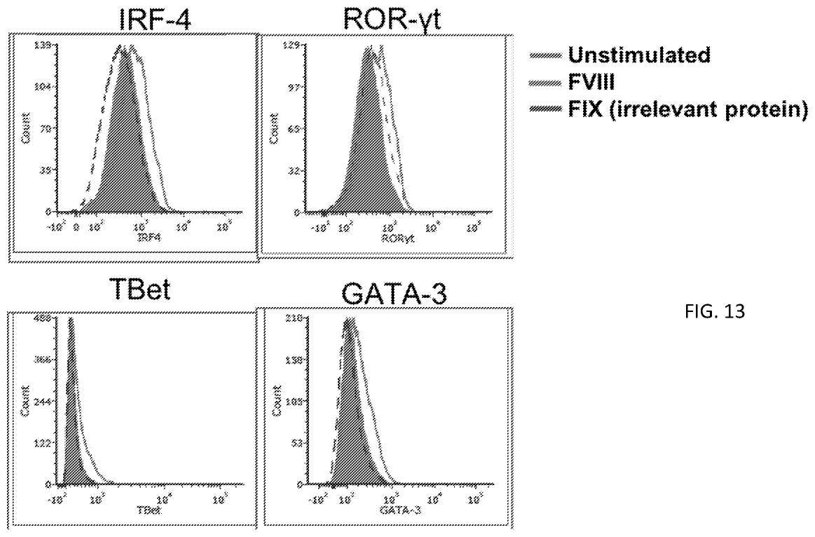

An analysis of transcription factors produced in CAR Tregs upon exposure to FVIII were tested. As shown in FIG. 13, in vitro FVIII stimulated CAR Tregs upregulated expression of the transcription factors IRF-4, ROR-γt, and GATA-3, but not Tbet.

As will be apparent to one of ordinary skill in the art from a reading of this disclosure, further embodiments of the present invention can be presented in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. Such equivalents are considered to be within the scope of this invention. Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention. The scope of the invention is as set forth in the appended claims and equivalents thereof, rather than being limited to the examples contained in the foregoing description. All publications mentioned herein, are incorporated by reference in their entirety. Nothing herein is to be construed as an admission that the invention is not entitled to antedate such disclosure by virtue of prior invention.

### REFERENCES

References listed below and throughout the specification are hereby incorporated by reference in their entirety.
Rogers G L, Herzog R W, Front Biosci 20: 256-603, 2015.
Markusic D M, Hoffman B E, Perrin G Q, Nayak S, Wang X, Loduca P A, High K A, Herzog R W. Effective gene therapy for haemophilic mice with pathogenic factor IX antibodies. EMBO Mol Med. 2013.
Chitlur M, Warrier I, Rajpurkar M, Lusher J M. Inhibitors in factor IX deficiency a report of the ISTH-SSC international FIX inhibitor registry (1997-2006). Haemophilia. 2009; 15(5):1027-31. [PubMed: 19515028]
Gouw S C, van den Berg H M, Oldenburg J, Astermark J, de Groot P G, Margaglione M, Thompson A R, van Heerde W, Boekhorst J, Miller C H, le Cessie S, van der Bom J G. F8 gene mutation type and inhibitor development in patients with severe hemophilia A: systematic review and meta-analysis. Blood. 2012; 119(12):2922-34. [PubMed: 22282501]
Halimeh S, Bidlingmaier C, Heller C, Gutsche S, Holzhauer S, Kenet G, Kurnik K, Manner D, Iorio A, Nowak-Gottl U. Risk factors for high-titer inhibitor development in children with hemophilia A: results of a cohort study. Biomed Res Int. 2013; 2013:901975. [PubMed: 24199202]
Mariani G, Siragusa S, Kroner B L. Immune tolerance induction in hemophilia A: a review. Semin Thromb Hemost. 2003; 29(1):69-76. [PubMed: 12640568]
Sarkar, D., Biswas, M., Liao, G., Seay, H. R., Perrin, G. Q., Markusic, D. M., Hoffman, B. E., Brusko, T. M., Terhorst, C., Herzog, R. W. (2014). Ex vivo expanded autologous polyclonal regulatory T cells suppress inhibitor formation in hemophilia. Mol. Ther. Methods. Clin. Dev. 1, pii: 14030.
Perdigoto, A. L., Chatenoud, L., Bluestone, J. A., Herold, K. C. (2016). Inducing and administering Tregs to treat human disease. Front. Immunol. 22(6), 654.
Carpenito, C., Milone, M. C., Hassan, R., Simonet, J. C., Lakhal, M., Suhoski, M. M. (2009). Control of large, established tumor xenografts with genetically retargeted human T cells containing CD28 and CD137 domains. Proc. Natl. Acad. Sci. 106, 3360-3365.
Brentjens, R. J., Riviere, I., Park, J. H., Davila, M. L., Wang, X., Stefanski, J., et al. (2011). Safety and persistence of adoptively transferred autologous CD19-targeted T cells in patients with relapsed or chemotherapy refractory B-cell leukemias. Blood 118, 4817-4828.
Porter, D. L., Levine, B. L., Kalos, M., Bagg, A., and June, C. H. (2011). Chimeric antigen receptor-modified T cells in chronic lymphoid leukemia. N. Engl. J. Med. 365, 725-733.
Kochenderfer, J. N., Dudley, M. E., Feldman, S. A., Wilson, W. H., Spaner, D. E., Maric, I., et al. (2012). B-cell depletion and remissions of malignancy along with cytokine-associated toxicity in a clinical trial of anti-CD19 chimeric-antigen-receptor-transduced T cells. Blood 119, 2709-2720.
Maude, S. L., Frey, N., Shaw, P. A., Aplenc, R., Barrett, D. M., Bunin, N. J., et al. (2014). Chimeric antigen receptor T cells for sustained remissions in leukemia. N. Engl. J. Med. 371, 1507-1517.
Ardouin L, Boyer C, Gillet A, Trucy J, Bernard A M, Nunes J, Delon J, Trautmann A, He, H T, Malissen B, and Malissen M (1999) Immunity, 10: 409-20.
Kochenderfer, J. N., Yu, Z., Frasheri, D., Restifo, N. P. and Rosenberg, S. A. (2010). Adoptive transfer of syngeneic T cells transduced with a chimeric antigen receptor that recognizes murine CD19 can eradicate lymphoma and normal B cells. Blood 116(19), 3875-3886.
Huston et al. (1988). Protein engineering of antibody binding sites: recovery of specific activity in an anti-digoxin single-chain Fv analogue produced in Escherichia coli, Proc. Natl. Acad. Sci. USA 85:5879-5883.
Bird et al. (1988). Single-chain antigen-binding proteins. Science 242:423-426.

What is claimed is:

1. A chimeric antigen receptor (CAR) specific for human clotting factor VIII (huF.VIII) comprising:
a single chain antibody variable region (scFv);
a CD28 signaling domain; and
a CD3 zeta signaling domain.

2. The CAR according to claim 1, wherein the scFv is derived from an antibody produced by a B cell of a subject that has developed huF.VIII inhibitors and wherein the scFv recognizes huF.VIII.

3. The CAR according to claim 1 or 2, wherein the scFv recognizes a portion of huF.VIII, which portion comprises a C1 domain and a C2 domain of huF.VIII, or fragments thereof.

4. The CAR according to any of claims 1-3, wherein the scFv recognizes amino acids 2125 to 2332 of huF.VIII.

5. The CAR according to claim 1, wherein the CD3 zeta signaling domain comprises six immune-receptor tyrosine-based activation motifs (ITAMs), each comprising a YXXL/I sequence, wherein X corresponds to a variable amino acids.

6. The CAR according to claim 5, wherein at least one of the six ITAMs comprises a substitution of the tyrosine with a non-tyrosine amino acid.

7. The CAR according to claim 6, wherein the non-tyrosine amino acids is selected from phenylalanine and tryptophan.

8. The CAR according to claim 6, wherein the first, second, fifth, and sixth ITAMs comprise a substitution of the tyrosine with a non-tyrosine amino acid.

9. A nucleic acid molecule comprising a nucleic acid sequence encoding a CAR according to any of claims 1-8.

10. A method for making a Treg cell expressing a CAR, the method comprising:
providing a Treg cell; and
introducing a nucleic acid molecule according to claim 9 into the Treg cell.

11. The method according to claim 10, further comprising introducing the nucleic acid molecule into a viral vector and introducing the viral vector into the Treg cell.

12. A regulatory T cell (Treg) expressing the CAR according to any of claims 1-8.

13. A regulatory T cell (Treg) expressing the CAR according to claim 5.

14. A composition comprising a regulatory T cell according to claim 12.

15. A composition comprising a regulatory T cell according to claim 13.

16. A method for inducing tolerance to huF.VIII protein therapy in a subject suffering from hemophilia A, the method comprising:
administering the composition according to claim 14 to the subject in an effective amount to induce tolerance to huF.VIII protein therapy.

17. A method for inducing tolerance to huF.VIII protein therapy in a subject suffering from hemophilia A and having been treated with huF.VIII protein therapy, the method comprising:
administering the composition according to claim 15 to the subject in an effective amount to induce tolerance to huF.VIII protein therapy.

18. The CAR of claim 1, wherein the a CD3 zeta signaling domain comprises a polypeptide sequence encoded by SEQ ID NO: 1 or SEQ ID NO:2.

19. The method of claim 11, wherein the viral vector is a lentiviral vector.

20. The method of claim 19, wherein the viral vector is encoded by a nucleic acid sequence comprising SEQ ID NO:3.

21. The method according to claim 16 or 17, wherein the composition is co-administered with huF.VIII protein therapy.


SEQUENCE LISTING (3 sequences)

SEQ ID NO: 1 - DNA, 342 bp, Artificial Sequence
  CD3 domain of a CD19-CAR
agagccaagttcagcagatccgccgagacagccgccaacctgcaggatcccaaccagctgttcaacgagctgaacctgggcagacgggaggaattcgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgttccagggcctgtccaccgccaccaaggacaccttcgacgccctgcacatgcagaccctggcccccagatga

SEQ ID NO: 2 - DNA, 342 bp, Artificial Sequence
  CAR signaling domain
agagccaagttttcccgctctgctgagacagccgctaacctgcaggatcctaaccagctgtacaacgagctgaacctgggcaggagagaggagtacgacgtgctggagaagaagcgggcccgcgatcctgagatgggaggcaagcagcagcggcgcaggaacccacaggagggagtgtacaacgccctgcagaaggacaagatggccgaggcttacagcgagatcggcacaaagggagagagacggcgcggcaagggacacgatggcctgtaccagggactgtccacagccaccaaagatacctatgatgccctccacatgcagacactcgcacccagataa

SEQ ID NO: 3 - DNA, 5989 bp, Artificial Sequence
  pMYs-IRES-GFP Retroviral Vector
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagaccccagcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattcctgcaggcctcgagggccggcgcgccgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcccagccacagtggtcgacgataaaataaaagattttatttagtctccagaaaaaggggggaatgaaagaccccacctgtaggtttggcaagctagcttaagtaagccattttgcaaggcatggaaaaatacataactgagaatagagaagttcagatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc